(19) United States
(12) Patent Application Publication
(10) Pub. No.: US 2013/0281926 A1
(43) Pub. Date: Oct. 24, 2013

(54) CANNULA FOR CARRYING OUT A REGIONAL EXTRACORPOREAL CIRCULATION

(71) Applicants: Maxime Raux, Paris (FR); Armelle Nicolas-Robin, Paris (FR)

(72) Inventors: Maxime Raux, Paris (FR); Armelle Nicolas-Robin, Paris (FR)

(73) Assignee: Assistance Publique - Hopitaux de, Paris (FR)

(21) Appl. No.: 13/806,492

(22) PCT Filed: Jun. 22, 2011
(86) PCT No.: PCT/EP2011/060425

§ 371 Date: Jun. 7, 2013

(30) Foreign Application Priority Data
Jun. 23, 2010 (FR) 1055029

(51) Int. Cl. A61M 1/36; A61B 17/12; A61M 25/00

(52) U.S. Cl. CPC A61M 1/3653; A61M 25/0026; A61B 17/12136
USPC 604/96.01

(57) ABSTRACT

The invention relates to a cannula for medical use, which is obstructed at the distal end thereof and which has an occlusion balloon enabling regionalization of the extracorporeal circulation, it being possible for said cannula to be multiperforated.

The invention relates to a novel arterial cannula which can be used to carry out a regional extracorporeal circulation, and which in particular is used for organ donation after cardiac death (DCD).

Organ donation after cardiac death (DCD) has been legal again in France since 2005.

The Biomedicines Agency has published the procedure to be followed when removing the kidneys and liver from donors of this type. This procedure involves the placement of arterial and venous femoral cannulas, in order to re-establish the normothermic circulation, and the placement of a Fogarty catheter by a contralateral femoral arterial route, which catheter is deployed and its balloon positioned approximately 5 cm above the diaphragm in order to regionalize the extracorporeal circulation and thereby avoid cardiac and cerebral reperfusion.

The procedure stipulates that circulatory assistance be set up after the positioning of the Fogarty catheter. This circulatory assistance must be operational 150 minutes after the cardiac arrest of the patient.

If this deadline, which is very short and sometimes impossible to achieve in practice, cannot be met, the procedure has to be stopped and the organs cannot be removed.

The average time of arrival of potential donors after cardiac death is 113±31 minutes (D. Barouk et al. Réanimation 2010 (19) 1S: SP 105). The procedure for introducing the Fogarty catheter takes approximately 20 to 30 minutes alone. It is then necessary to introduce the two cannulas in order to establish the regionalized circulation, which takes about another 20 to 30 minutes in the absence of technical difficulties. Thus, from the start of the intervention, a minimum period of 40 minutes is needed before being able to begin removing the organs. At this stage of the procedure, an average period of 153 minutes has therefore elapsed since the cardiac arrest, provided there have been no technical difficulties when inserting the cannulas.

It will therefore be appreciated that the speed of intervention and the speed of introducing the elements needed for circulatory assistance are crucial in organ donation after cardiac death (DDC).

The invention thus relates to an arterial cannula which is closed at its distal end and which is provided with an occlusive balloon permitting the regionalization of the extracorporeal circulation. In a preferred embodiment, this cannula is multi-perforated, which permits perfusion of the organs that are to be removed (kidneys and liver).

The use of this cannula thus makes it possible, in a single step, to install the element permitting the regionalization of the normothermic circulation and also the source for supply of oxygenated blood. It is thus possible to speed up the placement of the pre-requisites before the organs are removed, and therefore to improve the physiological state of the transplants by reducing the hot ischemia time, while at the same time reducing the failure rate currently observed as a result of the impossibility of observing the deadlines imposed on the establishment of the normothermic circulation. Finally, the use of this cannula makes it possible to avoid the incision in the contralateral femoral artery of the potential donor, thus enhancing the integrity of the body.

Cannulas that are coupled to occlusive elements already exist.

The document WO 99/37202 describes a cannula for maintaining blood flow in a patient whose heart has stopped, an occlusion element, allowing the ascending aorta to be obstructed in order to maintain the regionalization of the circulation. This cannula is open at its distal end and also has lateral ports located in proximity to the distal end of the cannula, in order to permit the circulation of the blood if the end is obstructed by the occlusion element.

The document WO 99/30766 describes a cannula coupled to an occlusive element located at the distal end thereof. This cannula can comprise three tubes (or lumens) for a) injecting a cardioplegia solution, b) injecting a fluid to inflate the occlusive element, and c) being used for aspiration. The solution supplied can be delivered upstream or downstream of the occlusive element.

US 2004/102734 describes two embodiments of cannulas having a balloon at their distal end, and also orifices located proximally with respect to said balloon. These devices are envisioned for use in cardiac surgery. These cannulas are thus intended to be used via the aorta. The cannula of the first embodiment (described in FIG. 1 of D1) has three lumens. The cannula of the second embodiment (illustrated in FIGS. 6 and 7 of D1) has two lumens. However, the two lumens have open proximal and distal ends (FIG. 7, and paragraph [0071]).

U.S. Pat. No. 6,267,747 describes cannulas having a balloon at their distal ends, and also orifices located proximally with respect to said balloon. These cannulas are intended for cardiac surgery (paragraph “Field of the invention”). They must permit the introduction of a cardioplegic liquid downstream of the occlusion element (column 2, lines 25-44). The cannulas of D2 have at least three lumens (FIG. 2).

US 2005/171505 describes cannulas with three channels. These cannulas are not closed downstream of the occlusion element, as they have orifices (3) downstream of this element (abstract, and FIG. 8).

US 2003/229332 describes a cannula which is more particularly intended for withdrawal of an element (such as a kidney stone).

U.S. Pat. No. 4,892,519 describes a cannula having orifices downstream of the balloon.

None of these documents describes the use of a cannula in carrying out a regional extracorporeal circulation for organ donation after cardiac death (DCD).

### DESCRIPTION OF THE FIGURES

FIG. 1 shows a side view of a cannula according to the invention. This cannula has a proximal end (1) and a distal end (2) that are connected by a body (3) comprising a first hollow tube (11) of which the proximal end (12) is open and the distal end (13) is closed, and a second hollow tube (21) of which the proximal end (22) and the distal end (23) are open, the distal end (23) of said tube (21) leading to the inside of a toric occlusion element (4) located in proximity to the distal end (2) of the cannula, said first hollow tube (11) having three orifices (14). In this specific case, the distal end (2) of the cannula has the shape of a rounded cone.

FIG. 2 shows plan views of the cannula according to the invention. In FIG. 2A, the two lumens (11) and (21) are concentric. In FIG. 2B, the lumen (21) is located on the edge of the body (3).

FIG. 3 shows a cannula according to the invention introduced into the aorta of a donor via the right femoral artery. The renal arteries (31, 32) and the celiac trunk (33) are indicated. Four orifices (14) located near the proximal end (2) are shown.

FIG. 4 shows a side view of another cannula according to the invention. It depicts the proximal end (1) and the distal end (2) that are connected by the body (3) comprising a first hollow tube (11) of which the proximal end (12) is open and the distal end (13) is closed, and a second hollow tube (21) of which the proximal end (22) and the distal end (23) are open, the distal end (23) of said tube (21) leading to the inside of an occlusion element (4) of cylindrical shape located in proximity to the distal end (2) of the cannula, said first hollow tube (11) having two orifices (14).

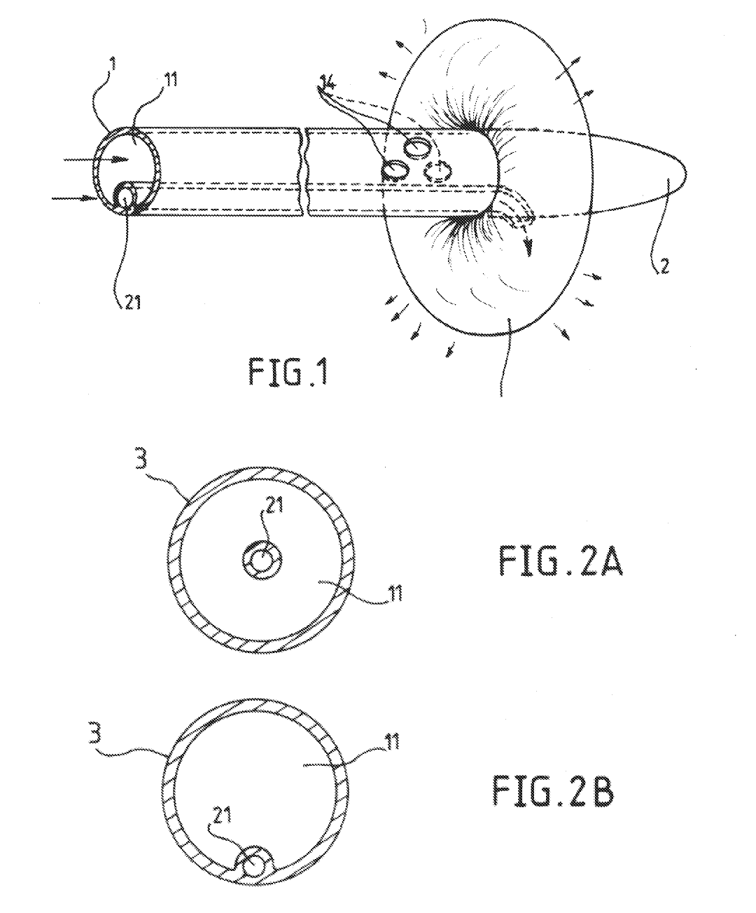

The invention thus relates to a cannula for medical use, comprising a proximal end (1) and a distal end (2) that are connected by a body (3) comprising a first hollow tube (11) of which the proximal end (12) is open and the distal end (13) is closed, and a second hollow tube (21) of which the proximal end (22) and the distal end (23) are open, the distal end (23) of said tube (21) leading to the inside of an occlusion element (4) located in proximity to the distal end (2) of the cannula, said first hollow tuber (11) having at least one orifice (14) arranged near the distal end (2) of the cannula and located proximally with respect to the occlusion element (4).

The orifice (14) thus connects the lumen (11) to the outside of the cannula.

Within the context of the present invention, the term “lumen” can be used in place of the term “tube”.

The terms “near the distal end” and “in proximity to the distal end” refer to a distance of between 1 and 10 cm from the distal end (2).

This use of the cannula (carrying out a regional extracorporeal circulation for organ donation after cardiac death (DCD)) therefore requires the presence of only two lumens: one lumen for inflating the inclusion element, and one lumen for re-establishing regionalized blood flow. It is thus clear that the cannula comprises only a lumen (11) and a lumen (21).

In a preferred embodiment, the distal end (2) of the cannula is of hemispherical shape. More preferably, this distal end (2) has the shape of a cone with a rounded point.

This shape of the distal end (2), and the fact that it narrows and has an internal diameter smaller than the diameter of the body (3) of the cannula, makes it possible to reduce the risk of damaging the arteries during the introduction of the cannula.

The occlusion element (4) can in particular be fixed to the cannula by a collar situated at the end of the cannula. This occlusion element is preferably a balloon and can be designated as such in the present description. When it is contracted, it lies against the outer surface of the body (3) of the cannula.

In a preferred embodiment, the occlusion element (4) is inflated by introduction of a fluid into this element via the second lumen (21) of the cannula. In this embodiment, a liquid solution is preferably used (such as physiological saline).

In other embodiments, the balloon (4) is filled with foam and is contracted, for insertion into the body of the patient, by application of a negative pressure or because it is covered by a sleeve. The balloon dilates, in order to obstruct the aorta, when the sleeve is withdrawn or when application of the negative pressure stops.

This occlusion element can be spherical, conical, elliptic, toric or funnel-shaped. The inner surface of the occlusion element (4) is adjacent to the outer surface of the body (3) of the cannula, the outer surface serving to occlude the aorta of the patient.

Some documents, for example U.S. Pat. No. 6,231,544, describe producing a cannula comprising a balloon.

In a preferred embodiment, this occlusion element (4) is placed approximately 2-3 cm before the distal end (2) of the cannula.

In a preferred embodiment, the occlusion element is said to be “compliant”, that is so say it adapts to the diameter of the artery once inflated (even when inflated to the maximum extent), without any risk of damaging the wall of the artery. Its maximum diameter after inflation is less than 45 mm. A compliant balloon is able to deform in order to adapt to the diameter of the aorta, even when inflated to the maximum extent.

It will be noted that the diameter of the aorta of patients is of the order of 24 mm for women and 24 to 27 mm for men.

The cannula according to the invention is intended in particular to be introduced via the femoral artery of a patient and inserted via the aorta until the occlusion element is located at the aortic orifice of the diaphragm.

The proximal end of each of the lumens is thus advantageously provided with a tubular connecting base for connecting the first lumen to a pump for injection of oxygenated blood and for connecting the second lumen to a pump or a syringe for inflating or deflating the occlusion element.

The first lumen (11) is thus intended to permit circulation of the oxygenated blood introduced from the proximal end. It is expedient that it can be released in the arteries of the patient in order to be able to continue irrigating the organs in question (liver and kidneys). It is thus expedient for the orifice or orifices (14) to permit a flowrate of the order of 4 liters per minute.

In a particular embodiment, the cannula has a plurality of orifices (14) distributed along the length of the first lumen (11). These orifices are preferably distributed all around the body (3) and are located at a distance of between 6 to 10 cm and 30 to 40 cm from the distal end (2) of the cannula.

As has been mentioned above, cardiac and cerebral reperfusion should be avoided. It is therefore clear that the orifices (14) intended to release the oxygenated blood introduced from the proximal end of the lumen (11) are all located proximally with respect to the occlusion element (4). It is also clear that there can be no orifice (14) (connecting the lumen (11) to the outside of the cannula) located distally with respect to the occlusion element (4), which would lead to reperfusion of the higher organs (heart or brain).

In a preferred embodiment, the internal diameter of the cannula is between 12 and 20 French (Fr). The French is the unit of measurement of the diameter of catheters and corresponds to 0.33 mm. In a preferred embodiment, the diameter of the cannula is between 15 and 18 Fr.

In order to maintain the blood flowrate at the outlet of the cannula via the orifice or orifices (14), it is preferable that the surface area of these orifices is equal to the surface area of the cannula. Thus, the surface area of the cannula is of the order of 20 mm² when the diameter of the cannula is 15 Fr. The total surface area of the orifices (14) must therefore be of this order. Thus, it is conceivable to provide 3 orifices (14) of the order of 3 mm in diameter, or 4 orifices (14) of the order of 2.5 mm in diameter, or 8 orifices (14) of approximately 1.8 mm in diameter. Another number of orifices (14) may be envisioned, their diameter then being easily calculated in order to meet the requirements in respect of the blood flowrate that has to be maintained in the arteries of the donor.

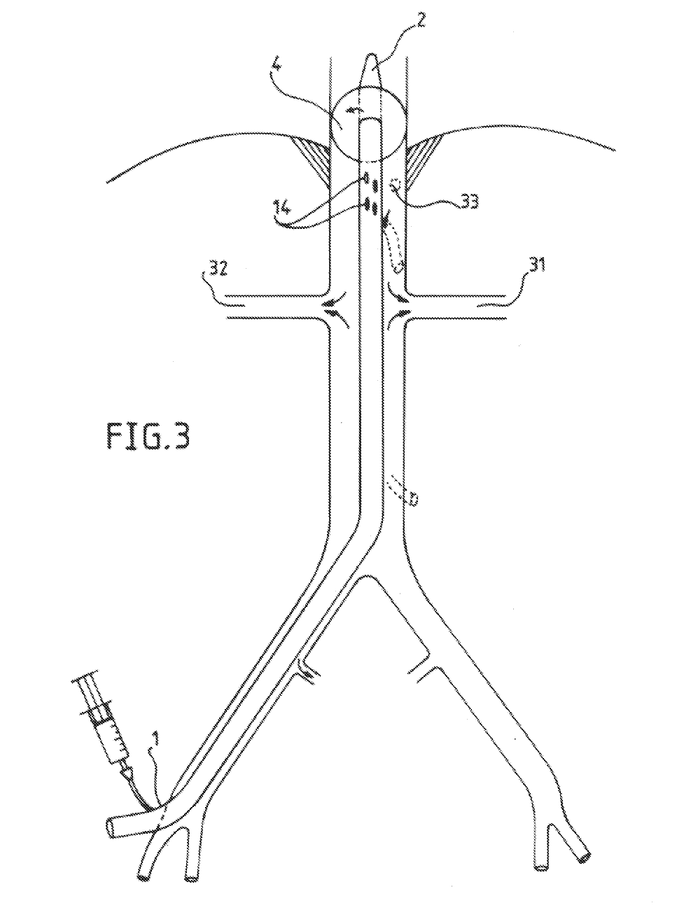

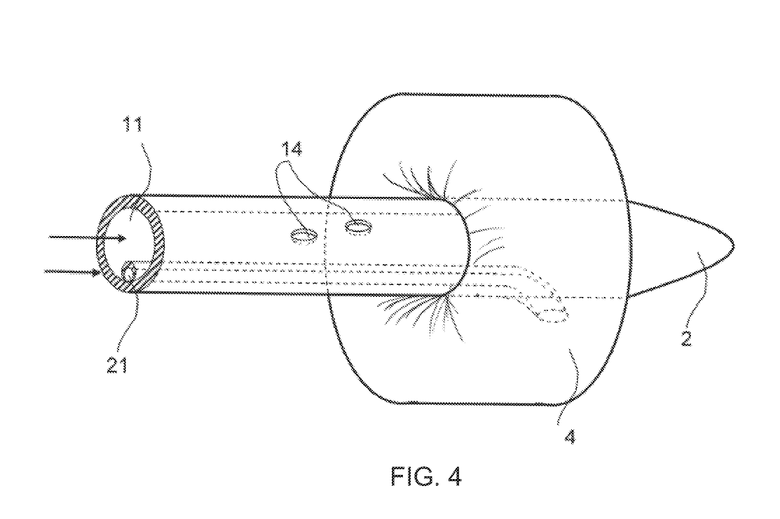

In a particular embodiment, the two tubes (11) and (21) are concentric. In this embodiment, the central tube (21) is used for circulation of the fluid intended to inflate the balloon, while the radial tube (11) is intended for the flow of blood (FIG. 2A).

In the preferred embodiment, the second tube (21) is situated on the periphery of the first tube (11), that is to say on an edge of the first tube, or directly inside the material constituting the body of the cannula (FIG. 2B).

In a preferred embodiment, the diameter of the first lumen (11) is 4 to 5 times greater than the diameter of the second lumen (21).

The cannula according to the invention must be inserted via the femoral artery of the patient as far as the level of the diaphragm. Provision is therefore made that it has a length greater than or equal to 50 cm, preferably about 80 cm.

The cannula according to the invention can be made of any material suitable for medical use. It is preferably made of non-thrombogenic and flexible plastic, so as to be able to pass easily through the arteries of the patient. It can thus be made of silicone, polyurethane or polytetrafluoroethylene (known under the trademark Teflon®).

The materials used to produce the occlusion element (4) are known in the art. This occlusion element can thus be made of polyurethane.

The cannula according to the invention can be sterilized by any means known in the art (for example using ethylene oxide or by beta or gamma irradiation) and packed in packages that are opened at the time of the operation.

As has been mentioned above, this cannula is used for carrying out a regional extracorporeal circulation and is preferably used in a procedure associated with removal of an abdominal organ such as the liver or the kidneys, particularly in organ donation after cardiac death.

One of the advantages of the cannula according to the invention is the possibility of perfusion of the oxygenated blood higher in the patient's body, and therefore closer to the arteries leading to the organs of interest (the celiac trunk leading to the liver, and the renal arteries). Indeed, with the system of the prior art, the arterial cannula releases the blood in the area of the branch between the iliac artery and the aorta. It therefore has to “climb” the aorta in order to enter the aforementioned arteries. The location of the orifices (14) directly under the occlusion device means that the blood is released nearer these arteries. This can therefore be an advantage for the quality of the organs.

The invention also relates to a method for removing an abdominal organ (liver, kidney) from a donor after cardiac death, said method comprising the steps of:
a. inserting the cannula according to the invention into a femoral artery, passing it into the aorta and positioning it in such a way that the occlusion element is located at the aortic orifice of the diaphragm,
b. introducing a venous cannula into a femoral vein of the patient,
c. deploying said occlusion element so as to block the circulation downstream of this element,
d. establishing a normothermic extracorporeal circulation by way of the cannula according to the invention and the venous cannula.

It is preferable to introduce the cannula into a femoral artery, this artery being easily accessible within the limited time available to the operator, but it can also be introduced into another artery, for example an external iliac artery. This also applies to the venous cannula mentioned in step c).

1. A cannula for medical use, comprising a proximal end (1) and a distal end (2) that are connected by a body (3) comprising a first hollow tube (11) of which the proximal end (12) is open and the distal end (13) is closed, and a second hollow tube (21) of which the proximal end (22) and the distal end (23) are open, the distal end (23) of said tube (21) leading to the inside of an occlusion element (4) located in proximity to the distal end (2) of the cannula, said first hollow tube (11) having at least one orifice (14) arranged near the distal end (2) of the cannula and located proximally with respect to the occlusion element (4), adapted for use in carrying out a regional extracorporeal circulation for organ donation after cardiac death (DCD).

2. A cannula for medical use, comprising a proximal end (1) and a distal end (2) that are connected by a body (3) comprising only a first hollow tube (11) of which the proximal end (12) is open and the distal end (13) is closed, and a second hollow tube (21) of which the proximal end (22) and the distal end (23) are open, the distal end (23) of said tube (21) leading to the inside of an occlusion element (4) located in proximity to the distal end (2) of the cannula, said first hollow tube (11) having a plurality of orifices (14) arranged along its length near the distal end (2) of the cannula and all located proximally with respect to the occlusion element (4).

3. The cannula as claimed in claim 1, characterized in that said first tube (11) has a plurality of orifices (14) along its length.

4. The cannula as claimed in claim 1, characterized in that the two tubes (11) and (21) are concentric.

5. The cannula as claimed in claim 1, characterized in that said second tube (21) is situated on the periphery of said first tube (11).

6. The cannula as claimed in claim 1, characterized in that the distal end (2) of the cannula has a hemispherical shape or the shape of a cone.

7. The cannula as claimed in claim 1, characterized in that the diameter of the first tube (11) is 4 to 5 times greater than the diameter of the second tube (21).

8. The cannula as claimed in claim 1, characterized in that said orifices (14) are located at a distance of between 6 and 40 cm from the distal end (2).

9. The cannula as claimed in claim 1, characterized in that it has a length greater than or equal to 50 cm.

10. The cannula as claimed in claim 1, characterized in that the internal diameter of the body (3) is between 3.96 and 6.6 mm.

11. The cannula as claimed in claim 1, characterized in that it is made of flexible plastic.

12. The cannula as claimed in claim 2, characterized in that the two tubes (11) and (21) are concentric.

13. The cannula as claimed in claim 2, characterized in that said second tube (21) is situated on the periphery of said first tube (11).

14. The cannula as claimed in claim 2, characterized in that the distal end (2) of the cannula has a hemispherical shape or the shape of a cone.

15. The cannula as claimed in claim 2, characterized in that the diameter of the first tube (11) is 4 to 5 times greater than the diameter of the second tube (21).

16. The cannula as claimed in claim 2, characterized in that said orifices (14) are located at a distance of between 6 and 40 cm from the distal end (2).

17. The cannula as claimed in claim 2, characterized in that it has a length greater than or equal to 50 cm.

18. The cannula as claimed in claim 2, characterized in that the internal diameter of the body (3) is between 3.96 and 6.6 mm.

19. The cannula as claimed in claim 2, characterized in that it is made of flexible plastic.
